(19) United States
(12) Patent Application Publication
(10) Pub. No.: US 2023/0414622 A1
(43) Pub. Date: Dec. 28, 2023

(54) PHARMACEUTICAL METHOTREXATE COMPOSITION

(71) Applicant: Leopold MTX GmbH, Berlin (DE)

(72) Inventor: Karl Skriner, Berlin (DE)

(73) Assignee: Leopold MTX GmbH, Berlin (DE)

(21) Appl. No.: 18/252,240

(22) PCT Filed: Nov. 9, 2021
(86) PCT No.: PCT/EP2021/081047

§ 371 Date: May 9, 2023

(30) Foreign Application Priority Data
Nov. 10, 2020 (DE) 10 2020 129 648.7

(51) Int. Cl. A61K 31/519; C07K 16/24; C07K 14/525; C07K 14/705; C07K 16/28; A61K 39/395

(52) U.S. Cl. CPC A61K 31/519; C07K 16/241; C07K 14/525; C07K 14/70521; C07K 16/2887; A61K 39/3955; C07K 2319/30; C07K 2317/21; C07K 2317/24

(57) ABSTRACT

Pharmaceutical compositions containing methotrexate or a pharmaceutically acceptable salt, hydrate, or derivative thereof, and a pharmaceutically active biopharmaceutical in a pharmaceutically acceptable solvent, are provided. Such compositions find medicinal use, inter alia, in the treatment of inflammatory autoimmune diseases such as rheumatoid arthritis.

### FIELD OF THE INVENTION

The present invention relates to pharmaceutical compositions comprising methotrexate or a pharmaceutically acceptable salt, hydrate or derivative thereof and a pharmaceutically active biopharmaceutical in a pharmaceutically acceptable solvent. The invention further relates to medical uses of the pharmaceutical compositions for the treatment of inflammatory autoimmune diseases, and to medical injection devices and drug containers containing such pharmaceutical compositions.

### BACKGROUND OF THE INVENTION

Methotrexate (abbreviated as MTX) is the nonproprietary name of the active pharmaceutical ingredient N-{4-[(2,4-diamino-6-pteridinylmethyl)methylamino]-benzoyl}-L-glutamic acid or (S)-2-{4-[(2,4-diaminopteridin-6-ylmethyl)methylamino]benzoylamino}-pentanedioic acid (IUPAC). Methotrexate is a folic acid antagonist and competitive inhibitor of the enzyme dihydrofolate reductase. Methotrexate is used as a basic therapy for many different inflammatory autoimmune diseases such as rheumatic diseases.

Therapeutic options for the treatment of autoimmune inflammatory diseases have been expanded by the development of specifically targeting biological agents known as biopharmaceuticals or biologics. Among these are strategies in which the biologics target specific neutralization of the proinflammatory cytokine tumor necrosis factor-α (TNF-α). Examples include the human anti-TNF-α monoclonal antibody adalimumab (trade name: Humira), the chimeric anti-TNF-α monoclonal antibody infliximab (trade name: Remicade), and the recombinant p75 TNF receptor Fc fusion protein etanercept (trade name: Enbrel). Other strategies aim to attenuate T- or B-lymphocyte activation with biologics that specifically bind to the B-lymphocyte antigen CD20 (e. g., rituximab, trade names: MabThera, Rituxan), the lymphocyte activation antigen CD80, or CD86 (e.g., abatacept, trade name: Orencia).

Biologics also represent a therapeutic breakthrough in oncology, in particular immunotherapies with biopharmaceutical immune checkpoint inhibitors, which show outstanding results even in advanced tumor diseases. Immune checkpoint inhibitors are usually monoclonal antibodies that specifically bind to immune checkpoints and thus prevent their signaling, especially anti-inflammatory signaling. An immune checkpoint is a protein that modulates the immune response, i. e. is either immune-suppressive or anti-inflammatory, or immune-stimulatory or pro-inflammatory, i. e. promotes inflammation. Immune checkpoints are involved in monitoring the proper function of the immune response, for example, by co-stimulating, i. e., positively regulating, or co-inhibiting, i. e., negatively regulating, the strength and intensity of an antigen-specific T-cell activation or T-cell effector function. For example, in some cancers, anti-inflammatory and co-inhibitory immune checkpoints, respectively, are upregulated in the course of immune evasion. Examples of established biologics in cancer treatment are nivolumab (trade name: Opdivo, manufacturer: Bristol-Myers Squibb), pembrolizumab (trade name: Keytruda; manufacturer: Merck/MSD Sharp & Dohme), pidilizumab (CT-011, manufacturer: CureTech Ltd.), avelumab (MSB0010718C, manufacturer: Pfizer), atezolizumab (MPDL3280A, Roche, Genentech), ipilimumab (MDX-010; trade name Yervoy, Bristol-Myers Squibb), and tremelimumab (ticilimumab, CP-675,206, Pfizer, MedImmune, Astra Zeneca).

In the state of the art, various combination therapies of methotrexate with immunosuppressive biologics are indicated when, for example, previous monotherapy with methotrexate has not been successful. For example, a combination of Humira with methotrexate is recommended by the European Drug Administration for the treatment of moderate to severe rheumatoid arthritis that has responded inadequately to disease-modifying antirheumatic drugs, including methotrexate monotherapy.

Such combination therapies must often be administered over a very long period of time and are therefore cost-intensive and complicated. In addition, the regular administration of high doses of active ingredients is perceived by subjects as very stressful and can lead to intolerance and increased side effects. Another problem with conventional combination therapies can be the generally sensitive physicochemical stability of the biologics, which leads to a continuous loss of quality with regard to the biological activity of the active agent and thus to reduced clinical efficacy, especially in ready-to-use formulations.

It is therefore an object of the present invention to provide an improved pharmaceutical composition suitable for medical use in the treatment of inflammatory autoimmune diseases, while at least partially solving the problems described above.

This object is solved by the subject matters of the independent claims. Further advantages and preferred embodiments of the invention can be derived from the dependent claims, the following description and the accompanying drawings.

### DESCRIPTION OF THE INVENTION

The present invention provides a pharmaceutical composition comprising a therapeutically effective amount of methotrexate or a pharmaceutically acceptable salt, hydrate or derivative thereof and a pharmaceutically acceptable solvent. Additionally, the composition includes a therapeutically effective amount of at least one pharmaceutically active biopharmaceutical that is at least partially, preferably for the most part or completely, dissolved in the solvent together with the methotrexate or pharmaceutically acceptable salt, hydrate or derivative thereof.

In this regard, the invention is based on the finding that in the composition according to the invention, methotrexate leads to a surprising improvement in the solubility, thermodynamic stability and binding affinity of the pharmaceutically active biopharmaceutical. This unexpected interaction of methotrexate and a biopharmaceutical in solution is substantiated in the detailed description of embodiments further below, to which reference is made here.

The inventor has recognized that the pharmaceutical composition thereby has particular advantages, especially for long-term therapy of inflammatory autoimmune diseases. Due to the improved solubility and thermodynamic stability of the biopharmaceutical, methotrexate and the biopharmaceutical can be provided and administered as a ready-to-use combination preparation. This significantly reduces the number of medications required and thus leads to improved therapy acceptance and a better quality of life for the subject, particularly in the case of parenteral administration. In addition, the reduction in the number of injections required also reduces the risk of infection from the syringes.

Since the methotrexate effectively counteracts denaturation of the biopharmaceutical in the composition according to the invention by its surprising stabilizing influence, it is simultaneously ensured that the biological activity of the biopharmaceutical in the liquid formulation is maintained over long periods of time. This allows the composition according to the invention to be provided, for example, in a form suitable and convenient for regular administration, for example as pre-filled syringes or injection pens with which the subject can advantageously self-administer the preparation, without compromising the pharmaceutical quality and clinical efficacy over a long period of time.

In addition, particular advantages result from the unexpected enhancement of the binding affinity of the biopharmaceutical by methotrexate. This results in an advantageous pharmacodynamic dose-response relationship of the composition according to the invention. As a result, it is possible in principle to dose the biopharmaceutical or even both active agents at a lower level than in the forms of administration commonly used to date. On the one hand, this leads to improved tolerability and fewer side effects of the pharmaceutical composition and, on the other hand, makes it possible to significantly reduce the costs in comparison to conventional combination therapies, particularly with regard to the generally high costs of the biopharmaceutical.

The effect of improved efficacy in the body can unfold particularly in the first hours of treatment. The composition according to the invention therefore offers particular advantages in the case of local administration into the inflamed tissue, joint, or tumor, which can be carried out, for example, by intrafocal, intraarticular or intratumoral injection. In addition, MTX in the composition according to the invention reduces the so-called anti-drug antibody (ADA) production. The effect of the reduced ADA leads to a higher stability of the biopharmaceutical in the body or locally in the inflamed tissue joint or tumor, which in turn can lead to a better therapy response. Repeated administration of the composition also allows higher and more consistent drug levels to be achieved, and, as a result, a better response from the subject is likely.

In accordance with the usual technical understanding of the term, derivatives of methotrexate are substances which have the same basic structure as methotrexate but have a different atom and/or atomic group in place of a hydrogen atom or a functional group, or in which one or more atoms and/or atomic groups have been removed, and which are therapeutically equivalent to MTX. Examples include a PEGylation, i. e., the methotrexate is chemically bonded to polyethylene glycol (PEG), and an acetylation, in which a hydrogen atom has been exchanged for an acetyl group on the basic structure of methotrexate. Another example is glycosylation. Such derivatizations can be used to increase the hydrate shell around the complex of MTX and biopharmaceutical and to increase the solubility of the biopharmaceutical. Other suitable derivatives of methotrexate are known to the skilled person.

Preferred derivatives of methotrexate are in particular those which are present in the pharmaceutically acceptable solvent as a zwitterion or are capable of forming a zwitterion in a, preferably physiological, solution. For this purpose, it is also possible to couple or substitute the basic structure of methotrexate with a functional group which is present in the pharmaceutically acceptable solvent as a zwitterion or is capable of forming a zwitterion in a, preferably physiological, solution.

A pharmaceutically acceptable salt of methotrexate is, for example, a sodium salt, especially a disodium salt (methotrexate disodium), or a calcium salt, as well as hydrates thereof.

The pharmaceutically acceptable solvent is not particularly limited. In principle, any pharmaceutically acceptable solvent can be used, which is known to the person skilled in the art for this purpose and which is not incompatible with the active ingredients or possibly other ingredients of the pharmaceutical composition. A preferred solvent is water, in particular water for injection purposes, which may contain isotonization additives. Another suitable solvent is saline solution, in particular an isotonic saline solution. Suitable isotonizing additives are known to the skilled person and may include, for example, soluble salts such as sodium chloride and/or potassium chloride, sugars such as glucose, lactose and/or trehalose, sugar alcohols such as mannitol and/or sorbitol, and combinations thereof.

Furthermore, the pharmaceutical composition may include excipients or additives that are commonly used in pharmaceutical solution formulation. In particular, the composition according to the invention may contain one or more of the following: buffers such as acetate, carbonate, phosphate or citrate buffers; antioxidants such as ascorbic acid, butylated hydroxytoluene or α-tocopherol; solubilizers and/or emulsifiers such as polyethylene glycol, polyethylene oxide, polysorbates, cyclodextrin, polyvidone, lecithin, glycocholate; acids; bases. Preferably, the pH-value of the pharmaceutical composition is between pH 7.0 and pH 9.0.

It is also possible that the pharmaceutical composition contains one or more preservatives. In principle, any preservative known to the skilled person and suitable for pharmaceutical purposes may be considered, for example PHB ester, benzalkonium chloride, benzyl alcohol, phenyl alcohol, cresol, thiomersal, as well as combinations thereof.

In preferred embodiments, the pharmaceutical composition does not contain any pharmaceutically active agents other than the methotrexate or pharmaceutically acceptable salt, hydrate or derivative thereof and the at least one pharmaceutically active biopharmaceutical.

Preferably, the pharmaceutical composition is in a form suitable for subcutaneous or intramuscular administration.

In one embodiment, the methotrexate or the pharmaceutically acceptable salt, hydrate or derivative thereof is provided at a concentration of from 2.5 milligrams per milliliter to 100 milligrams per milliliter or at a concentration of from 10 milligrams per milliliter to 50 milligrams per milliliter in the solvent.

In one embodiment, the methotrexate or the pharmaceutically acceptable salt, hydrate or derivative thereof is provided in the solvent at a concentration of from 5 milligrams per milliliter to 25 milligrams per milliliter.

In one embodiment, the methotrexate or the pharmaceutically acceptable salt, hydrate or derivative thereof is provided in the solvent at a concentration of from 25 milligrams per milliliter to 50 milligrams per milliliter.

In one embodiment, the methotrexate or the pharmaceutically acceptable salt, hydrate or derivative thereof is provided at a concentration of from 15 milligrams per milliliter to 40 milligrams per milliliter or at a concentration of from 20 milligrams per milliliter to 30 milligrams per milliliter in the solvent.

In a preferred embodiment, the methotrexate or the pharmaceutically acceptable salt, hydrate or derivative thereof is provided at a concentration of from 5 milligrams per milliliter to 50 milligrams per milliliter in the solvent.

In another embodiment, the methotrexate or the pharmaceutically acceptable salt, hydrate or derivative thereof is present in the solvent at a concentration of at most 50 milligrams per milliliter, at most 40 milligrams per milliliter, at most 30 milligrams per milliliter or at most 25 milligrams per milliliter.

It has been found that the methotrexate in the aforementioned concentrations has a particularly beneficial effect on the stability and binding affinity of the biopharmaceutical, so that the combination of both substances unfolds a particularly advantageous dose-response relationship in the composition according to the present invention.

In certain embodiments, the pharmaceutical composition may also comprise the methotrexate or the pharmaceutically acceptable salt, hydrate or derivative thereof at a concentration greater than 25 milligrams per milliliter.

In one embodiment, the methotrexate or the pharmaceutically acceptable salt, hydrate or derivative thereof is present in the solvent at a concentration of between 25 milligrams per milliliter and 150 milligrams per milliliter, preferably at a concentration of from 30 milligrams per milliliter to 100 milligrams per milliliter, more preferably at a concentration of from 40 milligrams per milliliter to 80 milligrams per milliliter.

In a preferred embodiment, the methotrexate or pharmaceutically acceptable salt, hydrate or derivative thereof is contained in the solvent at a concentration of from 50 milligrams per milliliter to 75 milligrams per milliliter, particularly at a concentration of about 50 milligrams per milliliter.

In one embodiment, the pharmaceutically active biopharmaceutical is provided in the solvent at a concentration of 1 milligram per milliliter to 150 milligrams per milliliter or at a concentration of 5 milligrams per milliliter to 100 milligrams per milliliter.

In one embodiment, the pharmaceutically active biopharmaceutical is provided in the solvent at a concentration of 10 milligrams per milliliter to 80 milligrams per milliliter or at a concentration of 15 milligrams per milliliter to 60 milligrams per milliliter.

In one embodiment, the pharmaceutically active biopharmaceutical is present in the solvent in a concentration of 20 milligrams per milliliter to 50 milligrams per milliliter.

In one embodiment, the pharmaceutically active biopharmaceutical is present in the solvent at a concentration of 25 milligrams per milliliter to 100 milligrams per milliliter.

In particular, the pharmaceutically active biopharmaceutical may be contained in the solvent at a concentration of 20 milligrams per milliliter to 50 milligrams per milliliter or at a concentration of 20 milligrams per milliliter to 30 milligrams per milliliter.

In further embodiments, the pharmaceutically active biopharmaceutical is provided in a concentration of at most 80 milligrams per milliliter, at most 70 milligrams per milliliter, at most 60 milligrams per milliliter, at most 50 milligrams per milliliter, at most 40 milligrams per milliliter, at most 30 milligrams per milliliter, or at most 25 milligrams per milliliter in the solvent.

At the aforementioned concentrations, the inventor found a particularly beneficial dose-response effect of the biopharmaceutical in combination with methotrexate.

Preferably, the pharmaceutically active biopharmaceutical is present in the pharmaceutical composition at a mass concentration that differs by less than 200%, by less than 100%, by less than 50%, by less than 40%, by less than 30%, by less than 20%, by less than 10%, or by less than 5% from the mass concentration of the methotrexate or the pharmaceutically acceptable salt, hydrate, or derivative thereof in the pharmaceutical composition.

Particularly preferred, the pharmaceutically active biopharmaceutical and the methotrexate or pharmaceutically acceptable salt, hydrate or derivative thereof are present in the pharmaceutical composition in the same or substantially the same mass concentration.

In one embodiment, the pharmaceutically active biopharmaceutical and the methotrexate or the pharmaceutically acceptable salt, hydrate, or derivative thereof are present in a mass ratio of 10:1 to 1:10 in the composition.

In another embodiment, the pharmaceutically active biopharmaceutical and the methotrexate or the pharmaceutically acceptable salt, hydrate, or derivative thereof are present in the composition in a mass ratio of 6:1 to 1:3 or 5:1 to 1:2.

In preferred embodiments, the pharmaceutically active biopharmaceutical and the methotrexate or the pharmaceutically acceptable salt, hydrate, or derivative thereof are present in a mass ratio of 3:1 to about 1:1 in the composition.

In certain embodiments, the mass proportion of the pharmaceutically active biopharmaceutical in the composition is greater than the mass proportion of the methotrexate or the pharmaceutically acceptable salt, hydrate, or derivative thereof.

Preferably, the pharmaceutical composition contains 2.5 milligrams per milliliter to 100 milligrams per milliliter of methotrexate or the pharmaceutically acceptable salt, hydrate or derivative thereof and 1 milligram per milliliter to 150 milligrams per milliliter of the biopharmaceutical. In a preferred embodiment, the pharmaceutical composition comprises 25 milligrams per milliliter to 50 milligrams per milliliter of methotrexate or the pharmaceutically acceptable salt, hydrate or derivative thereof and 20 milligrams per milliliter to 50 milligrams per milliliter of the biopharmaceutical. In another preferred embodiment, the pharmaceutical composition contains from 5 milligrams per milliliter to 25 milligrams per milliliter of methotrexate or the pharmaceutically acceptable salt, hydrate or derivative thereof and from 25 milligrams per milliliter to 100 milligrams per milliliter of the biopharmaceutical. In these concentration ranges, the inventor found a synergistic interaction between MTX and the biopharmaceutical, which was attributed inter alia to the fact that MTX increased the drug level and activity of the biologic.

In one embodiment, the pharmaceutically active biopharmaceutical is an immunosuppressive agent, i. e., a biological substance that reduces the functions of the immune system. For example, the pharmaceutically active biopharmaceutical can be a polypeptide or a protein having a binding domain for binding of a ligand or of an antigen, wherein the ligand or the antigen may be selected in particular from the group consisting of tumor necrosis factor-alpha (in short: TNF-α), B-lymphocyte antigen CD20 (in short: CD20), lymphocyte activation antigen CD80 (in short: CD80) or cluster of differentiation 86 (in short: CD86), and any combinations thereof.

In yet a further embodiment, the pharmaceutically active biopharmaceutical is an immune checkpoint inhibitor, i. e., a biologic that blocks receptor-ligand binding of an immune checkpoint. The immune checkpoint may be, e. g. a B7 protein or a receptor of a B7 protein, an MHC:peptide complex binding co-receptor, TNFRSF9 (Tumor Necrosis Factor Receptor Superfamily Member 9), CD40 (Cluster of Differentiation 40), TNFRSF4 (Tumor Necrosis Factor Receptor Superfamily Member 4), TNFRSF18 (Tumor Necrosis Factor Receptor Superfamily Member 18), CD27 (Cluster of Differentiation 27), TIGIT (T-cell immunoreceptor with Ig and ITIM domains), BTLA (B and T lymphocyte associated), or HAVCR2 (hepatitis A virus cellular receptor 2). In particular, the immune checkpoint may be selected from the group consisting of CD274 (Cluster of Differentiation 274), PDCD1LG2 (Programmed Cell Death 1 Ligand 2), PDCD1 (Programmed Cell Death 1), CD80 (Cluster of Differentiation 80), CTLA4 (Cytotoxic T-Lymphocyte-Associated Protein 4), ICOS (Inducible T-cell Co-Stimulator), CD276 (Cluster of Differentiation 276), C10orf54 (Chromosome 10 Open Reading Frame 54), HHLA2 (HERV-H LTR-associating 2), LAG3 (Lymphocyte-Activation Gene 3), CD160 (Cluster of Differentiation 160), KIR2DL4 (Killer cell Immunoglobulin-like Receptor, two domains, long cytoplasmic tail, 4), and KIR3DL1 (Killer cell Immunoglobulin-like receptor, three domains, long cytoplasmic tail, 1).

In preferred embodiments, the pharmaceutically active biopharmaceutical comprises an antibody, in particular a monoclonal antibody, or at least part of an antibody, in particular a Fab moiety, also referred to as a Fab fragment (fragment antigen binding), and/or a Fc moiety, also referred to as a Fc fragment (fragment crystallizable). Methotrexate has been found to have a particularly beneficial effect on the thermodynamic stability and binding affinity of these biologics. In this regard, reference is also made to the following detailed description of embodiments. Without limitation to theoretical considerations, the inventor presumes that methotrexate can cause a structural change by physicochemical interaction with the Fc moiety or Fab moiety, respectively, which leads to the improvement in the thermodynamic stability and binding affinity of these biologics by MTX that has been identified for the first time herein.

In a further embodiment, the pharmaceutically active biopharmaceutical is a bispecific antibody, i. e. an artificial immunoconjugate composed of components of two different monoclonal antibodies. Particularly in cancer immunotherapy, the known problems of bispecific antibodies, such as low efficacy due to an insufficient plasma half-life and increased immunogenicity, can be counteracted or at least partially remedied with the aid of the composition according to the invention.

In preferred embodiments, the pharmaceutically active biopharmaceutical is a TNF-α inhibitor, also referred to as a TNF-α blocker, that specifically binds to the proinflammatory cytokine TNF-α.

In one embodiment, the pharmaceutically active biopharmaceutical is adalimumab (trade name of the reference product: Humira). As the term is used herein, the term “adalimumab” also includes biosimilars of the reference product (trade names of biosimilars: e. g., Amsparity, Idacio, Imraldi, Halimatoz, Hefiya, Hyrimoz, Hulio, Amgevita). According to the usual understanding of the term in the art, a biosimilar is a biopharmaceutical that has no clinically relevant differences from the reference product and has equivalent safety and efficacy to the reference product.

In one embodiment, the pharmaceutically active biopharmaceutical is etanercept (trade name of the reference product: Enbrel). As the term is used herein, the term “etanercept” also includes biosimilars of the reference product (trade names of biosimilars: e. g. Nepexto, Benepali, Erelzi).

In one embodiment, the pharmaceutically active biopharmaceutical is Abatacept (trade name of the reference product: Orencia). As the term is used herein, the term “Abatacept” also includes biosimilars of the reference product.

In one embodiment, the pharmaceutically active biopharmaceutical is infliximab (trade name of the reference product: Remicade). As the term is used herein, the term “infliximab” also includes biosimilars of the reference product (trade names of biosimilars: e. g. Zessly, Flixabi, Inflectra, Remsima).

In one embodiment, the pharmaceutically active biopharmaceutical is golimumab (trade name of the reference product: Simponi). As the term is used herein, the term “golimumab” also includes biosimilars of the reference product.

In one embodiment, the pharmaceutically active biopharmaceutical is rituximab (trade name of the reference product: Mabthera). As the term is used herein, the term “rituximab” also includes biosimilars of the reference product (trade names of biosimilars: e.g., Ruxience, Blitzima, Ritemvia, Truxima, Rixathon, Riximyo).

In one embodiment, the pharmaceutically active biopharmaceutical is nivolumab (trade name of the reference product: Opdivo) or a biosimilar of the reference product.

In one embodiment, the pharmaceutically active biopharmaceutical is pembrolizumab (trade name of the reference product: Keytruda) or a biosimilar of the reference product.

In one embodiment, the pharmaceutically active biopharmaceutical is pidilizumab or a biosimilar thereof.

In one embodiment, the pharmaceutically active biopharmaceutical is avelumab (trade name of the reference product: Bavencio) or a biosimilar of the reference product.

In one embodiment, the pharmaceutically active biopharmaceutical is atezolizumab (trade name of the reference product: Tecentriq) or a biosimilar of the reference product.

In one embodiment, the pharmaceutically active biopharmaceutical is ipilimumab (trade name of the reference product: Yervoy) or a biosimilar of the reference product.

In one embodiment, the pharmaceutically active biopharmaceutical is tremelimumab, enoblituzumab, lirilumab, dacetuzumab, lucatumumab, urelumab, varlilumab, ulocuplumab, or a corresponding biosimilar thereof.

Of course, any combination of the aforementioned pharmaceutically active biologics is also possible. In preferred embodiments, the pharmaceutically active biopharmaceutical comprises adalimumab.

The present invention further relates to a medical use of the pharmaceutical composition described above in the treatment of an autoimmune inflammatory disease or a cancer disease of a subject.

In principle, the pharmaceutical composition may be administered orally or parenterally. Preferably, the administration is performed parenterally, in particular intrafocally, intraarticularly, intravenously, intratumorally, intrathecally, intracerebrally, intraperitoneally, intrapleurally, intralumbally, intralymphatically, intraocularly, intramuscularly and/or subcutaneously by injection. In one embodiment, the administration is intrafocal. In one embodiment, the administration is intraarticular. In one embodiment, administration is intravenous. In one embodiment, administration is intratumoral. In one embodiment, administration is intrathecal. In one embodiment, administration is intracerebral. In one embodiment, administration is intraperitoneal. In one embodiment, administration is intrapleural. In one embodiment, administration is intralumbar. In one embodiment, administration is intralymphatic. In one embodiment, administration is intraocular. In one embodiment, administration is intramuscular. In one embodiment, administration is subcutaneous.

In a preferred embodiment, the treatment comprises subcutaneous or intramuscular administration of the pharmaceutical composition. In this regard, a particular advantage is that no separate preparation of the active compounds by a physician or pharmacist is required. Therefore, it is possible that the administration is carried out by the subject itself, hereinafter also referred to as “self-administration”. The composition according to the present invention leads to a significant facilitation of self-administration compared to conventional combination therapies, in which the active compounds are applied separately. In this way, greater safety of the therapy carried out by self-application is achieved, which can also improve the success of the therapy.

In a further preferred embodiment, the treatment comprises local administration of the pharmaceutical composition. Preferably, the administration is intrafocal, i. e., local to an inflamed tissue, intraarticular, i. e., local to an inflamed joint such as a knee joint, wrist joint, or finger joint, intratumoral, i. e., local to a tumor, and/or intracethal, i. e., to a cerebrospinal fluid space around the brain or spinal cord, respectively. When applied locally, the increased efficacy of the composition according to the invention is particularly distinct compared to a separate systemic application of the active agents.

Alternatively, it is also generally possible to apply the methotrexate or the pharmaceutically acceptable salt, hydrate or derivative thereof and the biopharmaceutical locally separately, for example with separate syringes, so that the mixture of the two active compounds is performed just immediately in the inflamed tissue or joint. Thus, a variant of the present invention relates to a medical use of methotrexate or a pharmaceutically acceptable salt, hydrate or derivative thereof and a biopharmaceutical in the treatment of an inflammatory autoimmune disease or a cancer disease of a subject, which is characterized in that the methotrexate or the pharmaceutically acceptable salt, hydrate or derivative thereof is administered with a first injection and the biopharmaceutical is administered with a second injection, each intrafocally into an inflamed tissue or intraarticularly into an inflamed joint or intratumorally into a tumor or intrakethally into a cerebrospinal fluid space, in a manner such that within the tissue or joint or tumor or cerebrospinal fluid space, the biopharmaceutical is contacted with or mixed with the methotrexate or the pharmaceutically acceptable salt, hydrate or derivative thereof. The order of the first and second injections is not limited in this regard; for example, the biopharmaceutical may also be applied first followed by the methotrexate or the pharmaceutically acceptable salt, hydrate or derivative thereof. Simultaneous application is also possible. Preferably, the first injection and the second injection are administered within a time interval of less than five hours, less than four hours, less than three hours, less than two hours, or less than one hour. Preferably, the first injection and second injection are performed within a time interval of less than 50 minutes, less than 40 minutes, less than 30 minutes, less than 20 minutes, or less than 15 minutes. Particularly preferably, the first injection and second injection are performed within a time interval of less than ten minutes, less than five minutes, less than two minutes, or less than one minute. Most preferably, the first injection and second injection are performed substantially simultaneously. In this way, the combinatorial interaction of MTX and the biopharmaceutical established by the inventor can also unfold particularly advantageously in the form of a mixture of the active compounds generated in situ according to the invention. The invention further provides a kit for the aforementioned variant of the medical use. The kit comprises a first injection device having a fluid reservoir containing methotrexate or a pharmaceutically acceptable salt, hydrate or derivative thereof, and a second injection device having a fluid reservoir containing a pharmaceutically active biopharmaceutical. Preferably, the first injection device and the second injection device are configured for single injection or each contains a single dose of the respective active compound. Preferably, the kit contains instructions for its use.

In all other respects, the preferred embodiments of this medical use and kit, such as preferred concentrations of the respective active compound or dosages, correspond to those of the aspects of the invention already described above as well as below.

Due to the improved solubility and stability of the biopharmaceutical in the composition according to the present invention, it is possible to provide the combination preparation in a user-friendly, ready-to-use form suitable for long-term therapy. In preferred embodiments, administration is effected by means of a pre-filled syringe for single application or by means of a pen injector configured for multiple application of single doses. In this way, the subject can treat itself more easily and safely than with conventional combination therapies, which is a major advantage, particularly for patients with rheumatic autoimmune diseases and limited fine motor skills.

A pre-filled syringe for single application is, according to the usual understanding of the term in the art, a device, preferably sterile, with a fluid reservoir containing the pharmaceutical composition and an injection needle (cannula) through which the composition is applied to the subject. Further, a pre-filled syringe comprises mechanical means for forcing the pharmaceutical composition out of the fluid reservoir through the injection needle. A pre-filled syringe for single application is further characterized by containing a predetermined single dosage of the pharmaceutical composition, such that during administration the fluid reservoir must be completely discharged to administer the predetermined dosage.

Pen injectors for multiple application of single doses are also known in the field. Suitable pen injectors usually have a pen-shaped housing, which is equipped with a, optionally exchangeable, cartridge containing the pharmaceutical composition, and a cap with a short cannula. The desired individual dosage can be adjusted via an adjusting device such as a rotatable ring and applied to the subject via a triggering device such as a push button through the cannula.

Preferably, the pre-filled syringe or pen injector is configured to administer the pharmaceutical composition subcutaneously. For example, the cannula may have a length of no more than twelve millimeters, preferably no more than eight millimeters.

In a further preferred embodiment, the pre-filled syringe is configured to administer the pharmaceutical composition intra-articularly. For example, the cannula may have a length of at least 30 millimeters, preferably at least 40 millimeters.

For oral administration, the pharmaceutical composition is preferably encapsulated.

In one embodiment, the treatment comprises a dose regimen of one administration of the pharmaceutical composition every week. In another embodiment, one administration of the pharmaceutical composition every two weeks is provided, i. e., a biweekly administration.

Due to the advantageous pharmacodynamic properties of the composition according to the present invention, dose regimens in which the pharmaceutical composition is administered at most once in seven days, at most once in eight days, at most once in nine days, at most once in ten days, at most once in eleven days, at most once in twelve days, at most once in thirteen days, or at most once in fourteen days are also possible.

In one embodiment, the methotrexate or the pharmaceutically acceptable salt, hydrate or derivative thereof is administered in a dose or each a single dose of at least 2.5 milligrams, at least 5.0 milligrams, at least 7.5 milligrams, at least 10.0 milligrams, at least 12.5 milligrams, at least 15.0 milligrams, at least 17.5 milligrams, at least 20.0 milligrams, at least 22.5 milligrams, at least 25.0 milligrams, at least 27.5 milligrams, or at least 30.0 milligrams.

In one embodiment, the methotrexate or the pharmaceutically acceptable salt, hydrate, or derivative thereof is administered at a dose of at most 50.0 milligrams, at most 45.0 milligrams, at most 40.0 milligrams, at most 37.5 milligrams, at most 35 milligrams, at most 30 milligrams, at most 25 milligrams, at most 20 milligrams, at most 15 milligrams, at most 10 milligrams, or at most 7.5 milligrams per week.

Preferably, the methotrexate or the pharmaceutically acceptable salt, hydrate or derivative thereof is administered in a dose, or in a single dose, of 2.5 milligrams to 25 mg milligrams, 5 milligrams to 50 milligrams, 10 milligrams to 30 milligrams or 10 milligrams to 20 milligrams. In certain embodiments, the methotrexate or the pharmaceutically acceptable salt, hydrate or derivative thereof is administered in a dose or single dose, respectively, of 2.5 milligrams to 10 mg milligrams or 5 milligrams to 7.5 milligrams each. In particular, the foregoing values may refer to the dose per week or the dose per two weeks.

In one embodiment, the pharmaceutically active biopharmaceutical is administered in a dose, or in a single dose, of at least 2.5 milligrams, at least 5.0 milligrams, at least 7.5 milligrams, at least 10.0 milligrams, at least 12.5 milligrams, at least 15.0 milligrams, at least 17.5 milligrams, at least 20.0 milligrams, at least 22.5 milligrams, at least 25.0 milligrams, at least 27.5 milligrams, or at least 30.0 milligrams.

In one embodiment, the pharmaceutically active biopharmaceutical is administered in a dose of at most 150.0 milligrams, at most 125.0 milligrams, at most 100.0 milligrams, at most 80 milligrams, at most 60 milligrams, at most 50 milligrams, at most 40 milligrams, at most 37.5 milligrams, at most 35 milligrams, at most 30 milligrams, at most 25 milligrams, at most 20 milligrams, at most 15 milligrams, at most 10 milligrams, or at most 7.5 milligrams per week or per two weeks.

Preferably, the pharmaceutically active biopharmaceutical is administered in a dose, or in a single dose, of 2.5 milligrams to 80 milligrams, 5 milligrams to 50 milligrams, 10 milligrams to 30 milligrams, or 10 milligrams to 20 milligrams. In particular, the foregoing values may again refer to the dose per week.

In a preferred embodiment, the methotrexate or the pharmaceutically acceptable salt, hydrate, or derivative thereof is administered, particularly weekly or biweekly, in a dose, or in a single dose, of 2.5 milligrams to 25 milligrams, 10 milligrams to 20 milligrams, or 5 milligrams to 50 milligrams, and the pharmaceutically active biopharmaceutical of 5 milligrams to 50 milligrams.

Preferably, the methotrexate or the pharmaceutically acceptable salt, hydrate or derivative thereof is administered weekly in a dose, or in a single dose, of 2.5 milligrams to 25 milligrams or biweekly in a dose, or in a single dose, of 5 milligrams to 50 milligrams. The biopharmaceutical may also be administered in a pharmaceutically effective amount based on the subject's body weight.

Due to the combinatorial effect of the MTX and the biopharmaceutical in the composition according to the invention, doses as small as 5 milligrams to 7.5 milligrams of MTX biweekly can be sufficient to achieve the same therapeutic effect that is only achieved with doses of 10 milligrams to 20 milligrams of MTX biweekly when both active ingredients are administered conventionally. i. e. separately. At the same time, the higher efficacy results in fewer non-responders.

The composition according to the present invention has the advantage that the subjects have to be administered or injected with significantly smaller amounts of liquid overall compared to conventional combination therapy. In preferred embodiments, 0.5 milliliters to 3.0 milliliters, in particular 1.0 milliliters to 2.0 milliliters, of the pharmaceutical composition are administered per dose or per individual dose. Such small volumes simplify the application and are easier for the subject to tolerate, particularly in the case of subcutaneous injection.

In general, the pharmaceutical composition can be used for the treatment of any inflammatory autoimmune diseases that can also be reasonably treated with methotrexate or the pharmaceutically active biopharmaceutical. Non-limiting examples include rheumatoid arthritis, juvenile arthritis, psoriasis or psoriatic arthritis, systemic lupus erythematosus, systemic sclerosis, multiple sclerosis, Crohn's disease, ankylosing spondylitis, ulcerative colitis, polymyalgia rheumatica, vasculitis, non-infectious uveitis, acne inversa, Alzheimer's disease, and joint arthrosis. Other examples include polyarticular juvenile idiopathic arthritis, active enthesitis-associated arthritis, hidradenitis suppurativa, and Dupuytren's disease. In certain embodiments, the medical use of the composition according to the invention is for the treatment of rheumatoid arthritis.

The pharmaceutical composition can in principle be used for the treatment of any cancers for which the respective pharmaceutically active biopharmaceutical is also indicated. Non-limiting examples include a melanoma, a carcinoma, a sarcoma, a glioblastoma, a lymphoma, and/or a leukemia. The carcinoma may include, for example, an adenocarcinoma, a squamous cell carcinoma, a small cell carcinoma, a neuroendocrine carcinoma, a renal cell carcinoma, a urothelial carcinoma, a hepatocellular carcinoma, an anal carcinoma, a bronchial carcinoma, an endometrial carcinoma, a cholangiocellular carcinoma, hepatocellular carcinoma, testicular carcinoma, colorectal carcinoma, carcinoma of the head and neck, carcinoma of the esophagus, gastric carcinoma, mammary carcinoma, renal carcinoma, ovarian carcinoma, pancreatic carcinoma, prostatic carcinoma, thyroid carcinoma, and/or cervical carcinoma. A sarcoma may include, for example, angiosarcoma, chondrosarcoma, Ewing's sarcoma, fibrosarcoma, Kaposi's sarcoma, liposarcoma, leiomyosarcoma, malignant fibrous histiocytoma, neurogenic sarcoma, osteosarcoma, or rhabdomyosarcoma. For example, a leukemia may be acute myeloid leukemia (AML), acute lymphoblastic leukemia (ALL), chronic lymphocytic leukemia (CLL), or chronic myeloid leukemia (CML). A lymphoma may be Hodgkin's lymphoma or non-Hodgkin's lymphoma. A non-Hodgkin's lymphoma may be a B-cell lymphoma or a T-cell lymphoma. In particular, the malignancy is a malignant melanoma, which may be metastatic.

Another aspect of the present invention relates to an injection device for subcutaneous or intramuscular administration of a pharmaceutical composition. Herein, the injection device comprises a fluid reservoir containing a pharmaceutical composition as described above.

A preferred embodiment of the injection device is a pre-filled syringe containing a single dose of the pharmaceutical composition according to the invention.

Another preferred embodiment of the injection device is a pen injector configured to deliver a plurality of single doses of the pharmaceutical composition.

According to the description above, the composition according to the invention is particularly suitable and advantageous for the provision of a ready-to-use combination preparation for self-application by the subject, in particular due to the improved stability of the biopharmaceutical in combination with the methotrexate.

In a preferred embodiment, a single dosage contains about 2.5 milligrams to about 25 milligrams, about 10 milligrams to about 20 milligrams, or about 5 milligrams to about 50 milligrams of the methotrexate or the pharmaceutically acceptable salt, hydrate or derivative thereof and/or about 5 milligrams to about 50 milligrams of the pharmaceutically active biopharmaceutical. In this context, it is advantageous that in particular the cost-intensive biopharmaceutical can be dosed lower than in conventional combination therapies due to the improved thermodynamic stability and pharmacodynamic properties in the composition according to the invention while maintaining the same effect. In this way, not only the immunological side effects but also the therapy costs can be significantly reduced.

It is understood that the composition according to the present invention, due to its surprising properties, is also suitable and advantageous for ready-to-use preparations in other types of drug containers. Such a drug container may be, for example, a cartridge for a pen injector, a piercing vial, a vial, an infusion bag, an ampoule or a cartridge containing a pharmaceutical composition according to the embodiments set out above. In preferred embodiments, the pharmaceutical composition container is sterile and/or sealed.

Preferably, the pharmaceutical composition according to the present invention is used for filling or providing such injection devices and/or drug containers.

In a particularly preferred embodiment, the pharmaceutical composition according to the invention is contained in a nanoparticle or microparticle. Accordingly, the invention also relates to a nanoparticle and/or microparticle comprising the composition according to the present invention and to a medical use thereof for treating an inflammatory autoimmune disease or a cancer disease of a subject according to the above-described embodiments of the pharmaceutical composition of the present invention.

The provision or administration of the composition according to the invention in nanoparticles or microparticles has the great advantage that the release of the active compounds is continuous or substantially retarded in time. As a result, the increased efficacy of the composition according to the invention can be “preserved” in the particles and, at the moment of release, unfold directly and essentially undiminished locally, e. g. in a tumor or an inflamed joint, since no or hardly any dilution effect has occurred at this point compared to a conventional systemic application.

For example, the release of the pharmaceutical composition from the nanoparticles or microparticles can occur over at least 24 hours, at least 48 hours, at least 72 hours, at least 96 hours, at least seven days, at least 14 days, at least 21 days, at least 28 days, at least 40 days, at least 50 days, at least 60 days, at least 80 days, or more. In this way, a particularly safe and effective treatment, especially when administered locally, of inflammatory autoimmune disease and cancer can be achieved.

Suitable nano- and microparticles can be obtained, for example, from an amphiphilic codendrimer PGD of polyamidoamine and oligothylene glycol dendrons by antisolvent precipitation method. Such PGD particles can be loaded with the composition particularly efficiently due to an advantageous hydrophilicity, where e. g. loadings up to 50%, up to 60%, up to 70%, up to 80% or up to 85% (w/w) are possible. Other suitable nano- and microparticles are known to the skilled artisan or can be readily determined on the basis of this description.

Finally, the invention relates to a method for preparing a pharmaceutical composition according to the above. The method includes the following steps: Providing methotrexate or a pharmaceutically acceptable salt, hydrate or derivative thereof; providing at least one pharmaceutically active biopharmaceutical; at least partially dissolving the methotrexate or the pharmaceutically acceptable salt, hydrate or derivative thereof together with the at least one pharmaceutically active biopharmaceutical in a pharmaceutically acceptable solvent.

It is understood that preferred and advantageous embodiments of the pharmaceutical composition according to the invention, to the extent applicable, may also relate to the medical use of the pharmaceutical composition, the injection device and the medicament container, as well as the manufacturing method. Features disclosed above and below in connection with the pharmaceutical composition may therefore also relate to the medical use of the pharmaceutical composition, the injection device and the medicament container as well as the manufacturing method, and vice versa.

### BRIEF DESCRIPTION OF THE DRAWINGS

The invention will be explained in more detail below by way of exemplary embodiments with reference to the accompanying drawings. By no means is the invention intended to be limited to the exemplary embodiments and drawings. The drawings show:

FIG. 1 Temperature-dependent unfolding profiles of adalimumab (Humira) after 20 days of incubation in a composition according to the invention with MTX (A) and in a reference composition without MTX (B);

FIG. 2 the temperature stability of adalimumab (A), etanercept (B), abatacept (C), infliximab (D) and golimumab (E) each in a composition according to the invention with MTX compared to a reference composition without MTX;

FIG. 3 a dose response curve for determining the binding affinity of adalimumab to the ligand TNF-α in a composition according to the invention with MTX (A) compared to a reference composition without MTX (B).

### DETAILED DESCRIPTION OF EMBODIMENTS

For quality control and determination of the influence of methotrexate on the temperature stability of the biopharmaceutical in the pharmaceutical composition according to the invention, the thermal unfolding profiles of various biologics in compositions according to the invention were determined and compared with those of corresponding control compositions that did not contain MTX.

The following biologics were tested: Adalimumab (Humira) at a concentration of 25 milligrams per milliliter; etanercept (Enbrel) at a concentration of 25 milligrams per milliliter; abatacept (Orencia) at a concentration of 62.5 milligrams per milliliter; infliximab (Remicade) at a concentration of 16.5 milligrams per milliliter; and golimumab (Simponi) at a concentration of 33 milligrams per milliliter.

To prepare the compositions according to the invention, the biologics were combined with methotrexate disodium at concentrations of 25 milligrams per milliliter, 16.6 milligrams per milliliter, 10.0 milligrams per milliliter, 7.14 milligrams per milliliter, and 5.0 milligrams per milliliter, respectively. Phosphate buffered saline (PBS buffer) pH 7.5 was used as the solvent. As reference composition in each case served the biopharmaceutical in equivalent concentration in PBS buffer pH 7.5 without the addition of methotrexate disodium.

In each case, the above-noted concentrations were the final concentration of the biopharmaceutical or the methotrexate in the pharmaceutical composition.

After 20 days of incubation, the compositions were measured using a Tycho NT.6 fluorimeter (NanoTemper Technologies, Munich, Germany) according to the manufacturer's instructions to determine the thermal unfolding profiles of the biologics.

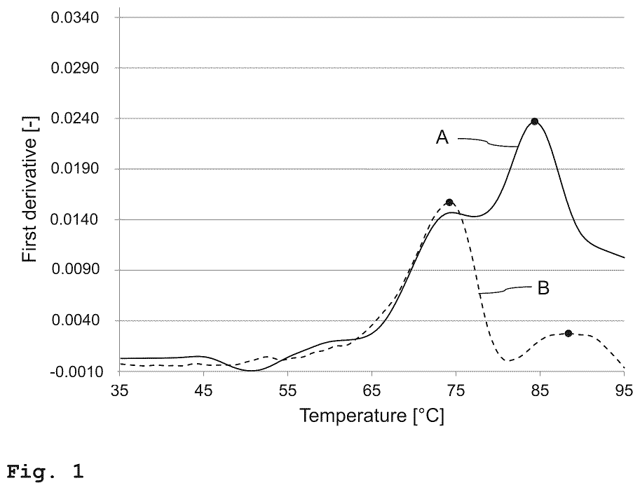

FIG. 1 shows an example of the unfolding profile as a function of temperature for the composition according to the present invention with 25 milligrams per milliliter adalimumab in combination with 25 milligrams per milliliter methotrexate disodium compared to the reference without MTX. The inflection point of each curve indicates the denaturation temperature of the biopharmaceutical in the composition. The denaturation temperature of adalimumab in the composition according to the invention was about 84° C., whereas the denaturation temperature in the reference composition without MTX was about 74° C. The temperature stability of adalimumab was thus increased by about 10° C. by the composition according to the invention. This corresponds to an increase of about 13.5%.

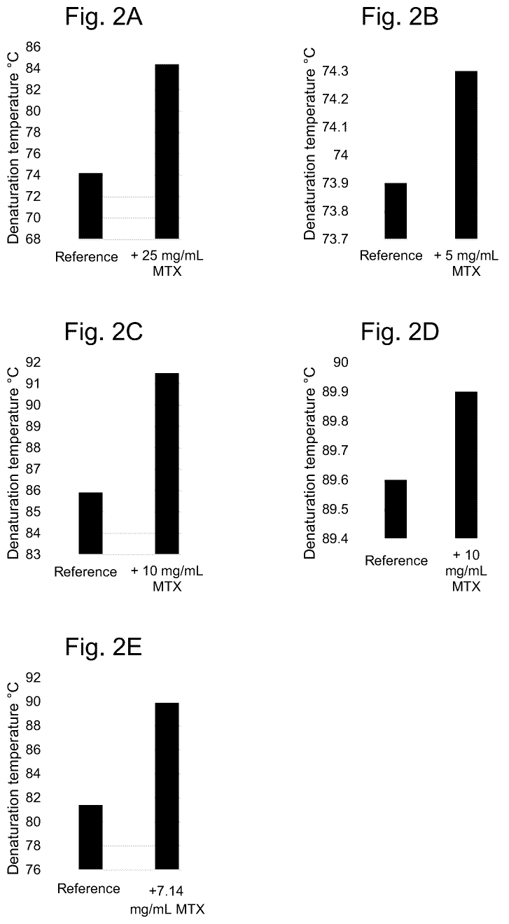

The summary of the results for the analysis of the temperature stability is shown in FIG. 2. For clarity, only the result for the composition according to the invention in which the greatest increase in temperature stability was determined compared to the reference composition is shown in each case. FIG. 2A shows that the temperature stability of adalimumab was increased from 74.2° C. to 84.4° C. by combination with MTX at a concentration of 25 milligrams per milliliter. FIG. 2B shows that the temperature stability of etanercept was increased from 73.9° C. to 74.3° C. by combination with MTX at a concentration of 5 milligrams per milliliter. FIG. 2C shows that the temperature stability of abatacept was increased from 85.9° C. to 91.5° C. by combination with MTX at a concentration of 10 milligrams per milliliter. FIG. 2D shows that the temperature stability of infliximab was increased from 89.6° C. to 89.9° C. by combination with MTX at a concentration of 10 milligrams per milliliter. FIG. 2E shows that the temperature stability of golilumab was increased from 81.4° C. to 89.9° C. by combination with MTX at a concentration of 7.14 milligrams per milliliter.

Accordingly, all tested biologics showed improved temperature stability in the compositions according to the invention compared to the reference compositions. From the improved stability at high temperatures, it can be directly concluded that the half-life of the biologics in the compositions according to the invention is significantly prolonged compared to the conventional compositions at moderate or low temperatures. In other words, the biopharmaceutical in the composition according to the invention is permanently better protected from degradation than in a conventional liquid formulation. As a result, the composition according to the invention is particularly advantageous to utilize as a ready-to-use combination preparation which, for example, in the form of a ready-to-use syringe or pen injector, ensures safe and effective application even after a long-term storage period.

In addition to thermodynamic stability, the binding affinity of the biopharmaceuticals in compositions according to the present invention was determined and compared with the binding affinity in corresponding reference compositions.

Binding studies were performed using microscale thermophoresis (MST) with the Monolith NT.115 device (NanoTemper Technologies, Munich, Germany).

Recombinant TNF-α, CD20, and CD80 (Acrobiosystems, USA) were labeled for MST measurements using the fluorescent dye Cy5 (Thermo Fisher Scientific, Germany) according to the manufacturer's instructions. The concentration of the recombinant proteins was measured after labeling using a UV-Vis spectrophotometer, and a labeling efficiency of 80 was determined.

For the MST measurements, 10 microliters each of the biopharmaceutical at a concentration of 16 to 27 millimoles per liter were mixed with 10 microliters each of a serial dilution of methotrexate disodium in PBS buffer pH 7.5 to prepare the compositions according to the invention. The final concentration of MTX in all samples was 55 micromoles per liter, while the concentration of the respective ligand ranged from 1.5 micromoles per liter to 25 millimoles per liter.

Samples were loaded into coated capillaries (NanoTemper Technologies) according to the manufacturer's instructions and fluorescence was recorded for 20 seconds at 100% laser power and 40% MST power. The temperature of the instrument was set at 25° C. for all measurements. After the recordal of MST time traces, the data were analyzed. The equilibrium dissociation constant (KD) was determined based on the change in normalized fluorescence as a function of the concentration of the corresponding ligand after 14 seconds of thermophoresis using MO affinity analysis software (NanoTemper Technologies).

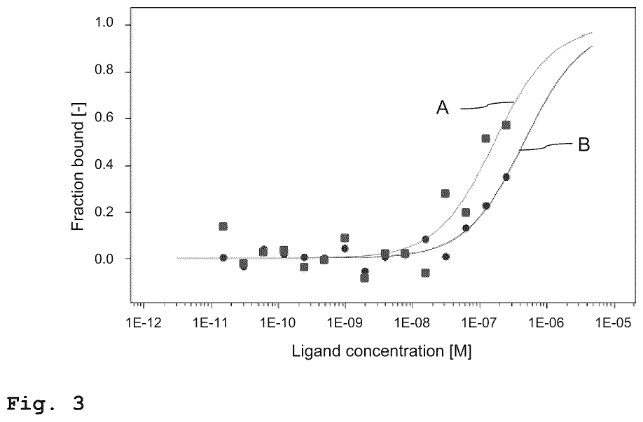

FIG. 3 shows an example of a dose-response curve from the analysis of the binding affinity of adalimumab to the ligand TNF-α in the composition according to the invention with MTX (A) compared to the control without MTX (B). Here, the equilibrium dissociation constant KD as a measure of the binding affinity of the antibody adalimumab to its antigen TNF-α corresponds to the concentration of TNF-α at which the half-maximal antibody:antigen complex formation between adalimumab and TNF-α is achieved. It is clearly evident that this half-maximal complex formation in the composition according to the invention occurs at significantly lower concentrations of the antigen compared to the control, corresponding to a lower KD value of adalimumab in the composition according to the invention and thus a higher binding affinity to TNF-α.

The following table summarizes the binding affinities of adalimumab, golimumab, infliximab and etanercept with respect to TNF-α and abatacept and rituximab with respect to CD20 and CD80, respectively. The values shown are the arithmetic mean values from six independent test series in each case.

| | KD without | | KD with | | Relative increase |
|---|---|---|---|---|---|
| Biologic | MTX (reference) | | MTX | | in binding strength |
| Golimumab | 25.3 | pM | 19.0 | pM | 33% |
| Infliximab | 59.0 | pM | 51.8 | pM | 14% |
| Etanercept | 11.6 | pM | 5.0 | pM | 132% |
| Adalimumab | 147.3 | pM | 139.7 | pM | 5% |
| Abatacept | 361.6 | pM | 292.0 | pM | 24% |
| Rituximab | 11.3 | nM | 6.8 | nM | 66% |

The results demonstrate a significant improvement in the binding strength of the tested biopharmaceuticals in the pharmaceutical compositions according to the present invention compared to the reference compositions that did not contain MTX.

It is understood that the improved binding affinity of the biopharmaceutical also leads to particular advantages of the composition according to the invention in medical application. In particular, the higher binding affinity results in improved pharmacodynamics of the biopharmaceutical, which manifests itself, for example, in a more efficient blocking of TNF-α in vivo, whereby the combination therapy with biopharmaceutical and MTX can develop a higher overall efficacy and reliability. As a result, fewer subjects may fail to respond or respond inadequately to therapy, or therapy may be more successful in responding subjects. In addition, due to the improved efficacy and increased stability of the biopharmaceutical, the composition according to the present invention makes it possible to reduce the dosage of the biopharmaceutical compared with conventional, separate administration without impairing the therapeutic effect. As a result, adverse immunological reactions due to anti-drug antibodies and ADA-induced adverse changes in the toxicology, pharmacokinetics, or efficacy of the biopharmaceutical, e. g., secondary incompatibilities or secondary non-response, can be reduced or avoided. In addition, the composition according to the invention can in this way also lead to a significant cost reduction in the therapy of inflammatory autoimmune diseases.

These advantages are particularly effective in local therapy, for example when the composition according to the present invention is applied directly intraarticularly into an inflamed or swollen joint, as in about 20% of cases of oligoarthritis.

The above results demonstrate a systematic interaction between MTX and biopharmaceutical in accordance with the invention. In this respect, the tests carried out herein as representative examples also provide sufficient indications that the advantageous properties of the composition according to the invention also arise with other biologics not tested separately here, such as immune checkpoint inhibitors or antibody-drug conjugates in cancer therapy.

The combinatorial effects of MTX and a biopharmaceutical found by the inventor in the composition according to the present invention with regard to the stability and efficacy of the substances and the advantages resulting therefrom for the therapy of inflammatory autoimmune diseases and cancer diseases could not have been expected from a skilled artisan's point of view.

The invention is not limited by the description of the exemplary embodiments. Rather, the invention encompasses any new feature as well as any combination of features, which in particular includes any combination of features in the patent claims, even if this feature or combination of features is not itself explicitly stated in the patent claims or the embodiment examples.

1: A pharmaceutical composition, comprising:
methotrexate or a pharmaceutically acceptable salt, hydrate or derivative thereof,
a pharmaceutically acceptable solvent, and
at least one pharmaceutically active biopharmaceutical, which is at least partially dissolved in the solvent in combination with the methotrexate or the pharmaceutically acceptable salt, hydrate or derivative thereof.

2: The pharmaceutical composition according to claim 1, wherein the methotrexate or the pharmaceutically acceptable salt, hydrate or derivative thereof is present in the solvent at a concentration of from 2.5 milligrams per milliliter to 100 milligrams per milliliter or at a concentration of from 5 milligrams per milliliter to 50 milligrams per milliliter.

3: The pharmaceutical composition according to claim 1, wherein the methotrexate or the pharmaceutically acceptable salt, hydrate or derivative thereof is present in the solvent at a concentration of from 5 milligrams per milliliter to 25 milligrams per milliliter or at a concentration of from 25 milligrams per milliliter to 50 milligrams per milliliter.

4: The pharmaceutical composition according to claim 1, wherein the at least one pharmaceutically active biopharmaceutical is present in the solvent at a concentration of from 1 milligram per milliliter to 150 milligrams per milliliter or at a concentration of from 5 milligrams per milliliter to 100 milligrams per milliliter.

5: The pharmaceutical composition according to claim 1, wherein the at least one pharmaceutically active biopharmaceutical is present in the solvent at a concentration of from 20 milligrams per milliliter to 50 milligrams per milliliter or at a concentration of from 25 milligrams per milliliter to 100 milligrams per milliliter.

6: The pharmaceutical composition according to claim 1, wherein the at least one pharmaceutically active biopharmaceutical is an immunosuppressive agent.

7: The pharmaceutical composition according to claim 1, wherein the at least one pharmaceutically active biopharmaceutical is a tumor necrosis factor-α inhibitor.

8: The pharmaceutical composition according to claim 1, wherein the at least one pharmaceutically active biopharmaceutical is a polypeptide or a protein having a binding domain for binding of a ligand or an antigen.

9: The pharmaceutical composition according to claim 8, wherein the ligand or antigen is selected from the group consisting of tumor necrosis factor-α, B lymphocyte antigen CD20, lymphocyte activation antigen CD80, cluster of differentiation 86 (CD86), and any combinations thereof.

10: The pharmaceutical composition according to claim 1, wherein the at least one pharmaceutically active biopharmaceutical is selected from the group consisting of adalimumab, etanercept, abatacept, infliximab, golimumab, rituximab, and any combinations thereof.

11: A method of treatment of an autoimmune inflammatory disease, comprising:
administering an effective amount of the pharmaceutical composition according to claim 1 to a subject in need thereof.

12: The method according to claim 11, wherein that the treatment comprises a subcutaneous or intramuscular administration of the pharmaceutical composition performed by the subject itself.

13: The method according to claim 11, wherein the treatment comprises administration by a single-dose pre-filled syringe or by a pen-injector configured for multiple-dose administration of single doses.

14: The method according to claim 11, wherein the treatment comprises local administration of the pharmaceutical composition.

15: The method according to claim 11, wherein the treatment comprises intrafocal or intraarticular administration of the pharmaceutical composition.

16: The method according to claim 11, wherein the treatment comprises an oral administration of the pharmaceutical composition.

17: The method according to claim 11, wherein the methotrexate or the pharmaceutically acceptable salt, hydrate or derivative thereof is administered at a dose of from 2.5 milligrams to 25 milligrams per week.

18: The method according to claim 11, wherein the at least one pharmaceutically active biopharmaceutical is administered at a dose of from 5 mg to 50 mg per week.

19: The method according to claim 11, wherein the autoimmune inflammatory disease is selected from the group consisting of rheumatoid arthritis, juvenile arthritis, psoriatic arthritis, systemic lupus erythematosus, systemic sclerosis, multiple sclerosis, Crohn's disease, ankylosing spondylitis, ulcerative colitis, polymyalgia rheumatica, vasculitis, and any combinations thereof.

20: The method according to claim 11, wherein the inflammatory disease is rheumatoid arthritis.

21: An injection device for subcutaneous or intramuscular administration of a medicament, wherein the injection device comprises a fluid reservoir containing the pharmaceutical composition according to claim 1.

22: The injection device according to claim 21, wherein the injection device is a pre-filled syringe containing a single dose of the pharmaceutical composition.

23: The injection device according to claim 21, wherein the injection device is a pen injector configured for delivering a plurality of single doses of the pharmaceutical composition.

24: The injection device according to claim 22, wherein the single dose comprises 2.5 milligrams to 25 milligrams or 5 milligrams to 50 milligrams of the methotrexate or the pharmaceutically acceptable salt, hydrate or derivative thereof, and/or 5 milligrams to 50 milligrams of the at least one pharmaceutically active biopharmaceutical.

25: A cartridge for a pen injector, wherein the cartridge comprises the pharmaceutical composition according to claim 1.

26: A sterile and/or sealed drug container, selected from the group consisting of a piercing vial, a vial, an infusion bag, a glass ampoule and a cartridge, wherein the drug container contains the pharmaceutical composition according to claim 1.

27: A drug-loaded nanoparticle or microparticle, wherein the nanoparticle or microparticle comprises the pharmaceutical composition according to claim 1.

28: A method for providing the injection device according to claim 21, the method comprising:
filling the injection device with the pharmaceutical composition according to claim 1.

29: A method of preparing the pharmaceutical composition according to claim 1, comprising:
providing methotrexate or a pharmaceutically acceptable salt, hydrate or derivative thereof,
providing at least one pharmaceutically active biopharmaceutical, and
at least partially dissolving the methotrexate or the pharmaceutically acceptable salt, hydrate or derivative thereof together with the at least one pharmaceutically active biopharmaceutical in a pharmaceutically acceptable solvent.

30: A method for treating an inflammatory autoimmune disease of a patient with a drug combination comprising
methotrexate or a pharmaceutically acceptable salt, hydrate or derivative thereof, and
a pharmaceutically active biopharmaceutical,
the method comprising:
administering the methotrexate or the pharmaceutically acceptable salt, hydrate or derivative thereof with a first injection, and
administering the pharmaceutically active biopharmaceutical with a second injection different from the first injection, intrafocally into an inflamed tissue or intraarticularly into an inflamed joint, wherein administration is performed such that within the tissue or joint the pharmaceutically active biopharmaceutical is contacted with the methotrexate or the pharmaceutically acceptable salt, hydrate or derivative thereof, to yield the drug combination.

31: The method according to claim 30, wherein the first and second injections are administered less than one hour apart in time.

32: A kit for the treatment of an inflammatory autoimmune disease of a subject with the method according to claim 30, wherein the kit comprises:
a first injection device having a fluid reservoir containing the methotrexate or the pharmaceutically acceptable salt, hydrate or derivative thereof, and
a second injection device separate from the first injection device, having a fluid reservoir containing the pharmaceutically active biopharmaceutical.
